(19) United States
(12) Patent Application Publication
(10) Pub. No.: US 2007/0014881 A1
(43) Pub. Date: Jan. 18, 2007

(54) Cosmetic formulations and skin treatment

(76) Inventor: Ingrid Harder-Tolar, Bodega Bay, CA (US)

Correspondence Address: EDWARD S. SHERMAN, ESQ., 3554 ROUND BARN BLVD., SUITE 303, SANTA ROSA, CA, 95403, US

(21) Appl. No.: 11/190,435

(22) Filed: Jul. 27, 2005

Related U.S. Application Data
(60) Provisional application No. 60/699,276, filed on Jul. 13, 2005.

(51) Int. Cl. A61K 36/48; A61K 36/87; A61K 36/63

(52) U.S. Cl. 424/757; 424/766; 424/769

(57) ABSTRACT

A process of skin treatment to soften the skin and minimize wrinkles utilizes in the first step the spray application of a mist to the skin. This first step is followed by the application of a mixture of beneficial vegetable oils that with essential oils. The first step of the treatment facilitates the absorption of the beneficial oils in the second step. As the composition and process also provides for the firming and toning of breast tissue, it promotes a daily regimen of efficient examination critical in the early self-discovery of abnormal tissue, which may be malignant. The preferred compositions are optimized to facilitate the breast self-examination process, providing an appropriate level of lubricity for an extended time without an oily residue.

### CROSS REFERENCE TO RELATED APPLICATIONS

The present application claims priority to provisional application for “Cosmetic Formulations and Skin Treatment “having Ser. No. 60/699,276, filed with the U.S. Patent and Trademark Office on Jul. 13, 2005 under Attorney Docket #163.01, which is incorporated herein by reference.

### BACKGROUND OF INVENTION

The present invention relates to a cosmetic treatment for skin tissue, and in particular the treatment of the skin tissue of the breast.

It is known that essential oil provide effective cosmetic treatments for skin tissue. For example, U.S. Pat. No. 6,574,504 (to Mazaury et al., issued Jun. 3, 2003), which is incorporated herein by reference, teaches that for skin wrinkles, one or more essential oils can for example be used, chosen from the essential oils of bitter almond, spike lavender, basil, benzoin, rosewood, cade, cajeput, camomile, cinnamon, carrot, cedar, lemon, copaiba, cypress, elemi, eucalyptus, juniper, geranium, clove, helichrysum, hyssop, lavender, lime, mace, nutmeg, niaouli, bitter orange, orange, oregano, palmarosa, grapefruit, patchouli, rosemary, rose, sandalwood, savory, sage, thyme, ylang-ylang, et Canada balsam, balsam of Peru and balsam of Tolu. The '504 patent further teaches that essential oils used in preference are basil, benzoin, rosewood, cajeput, camomile, carrot, cedar, cypress, geranium, lavender, niaouli, bitter orange, oregano, palmarosa, patchouli, rosemary, sandalwood, sage, thyme and ylang-ylang, notably basil, rosewood, cajeput, camomile, carrot, cypress, geranium, lavender, niaouli, bitter orange, palmarosa, patchouli, rosemary, sage, thyme and ylang-ylang and particularly basil, rosewood, carrot, cypress, geranium, lavender, bitter orange, palmarosa, patchouli, rosemary, sage and thyme. The '504 patent also teaches that for breast problems, as for firming of the bust, one or more of the same essential oils can be used, with a similar preference particularly for bitter almond, rosewood, cedar, lemon, eucalyptus, clove, hyssop, lime, mace, nutmeg, bitter orange, palmarosa, rosemary and sandalwood.

U.S. Pat. No. 6,544,534 (to Malmgren et al., issued Apr. 8, 2003) which is incorporated herein by reference, discloses the use of a skin softener that includes include sea salt, Epsom salt, almond oil, apricot kernel oil, avocado oil, jojoba oil, aloe vera gel, castor oil, vitamin E, vegetable glycerin and soap. The '534 patent further discloses that essential oils may optionally be included in the conditioner to add fragrance.

Further, U.S. Pat. No. 5,916,573 (to Spiers, et al., issued Jun. 29, 1999), which is incorporated by reference, discloses a topical treatment of the skin with a grapeseed oil composition that comprises about 1 to about 99% by weight of grapeseed oil; b) at least one hydrating agent; and c) water. Hydrating agents include vegetable glycerin, aloe-vera, and vegetable oils other than grapeseed oil, for example, vitamin E oil, jojoba oil, flaxseed oil, primrose oil and any other botanical oil.

A high percentage of jojoba oil may be extracted form the jojoba plant. The jojoba plant is a perennial evergreen shrub endemic to the Sonoran desert (south west Arizona and California, Northern Mexico and Baja California), having the scientific name Simmondsia chinensis. Jojoba wax is a straight-chain ester of mono-unsaturated long chain fatty acids and fatty alcohols having an average total carbon chain length of 42 carbons. The Jojoba oil is a light yellow liquid room temperature. Although it does not become rancid or damaged by prolonged exposure to high temperatures, it can be isomerized, hydrogenated, sulphurized, chlorinated or transesterified. The oil and its wax like derivatives have a wide range of industrial uses, including skin care preparations such as lotions, moisturizers, massage oils, and soothing creams.

Further, U.S. Pat. No. 5,266,318 (to Taylor-McCord, issued on Nov. 30, 1993), which is incorporated herein by reference, discloses a skin therapeutic mixture containing cold-processed aloe vera extract having the yellow sap and aloin removed. The above mixture is disclosed as useful for treatment of irradiated skin, open sores, wounds and abrasions. The '318 patent discloses compositions that include that include jojoba wax (2%) and the essential oil of lavender (0.05%).

### SUMMARY OF INVENTION

A first object of the present invention to provide a formulation and method of treating skin to remove wrinkles, increase firmness and soften the skin.

A further object of the invention is to provide an improved method of inspecting breast tissue that promotes self-examination on a daily basis.

It is another object of the invention to provide a skin beneficial formulation in a manner conducive to the use on breast tissue, in a manner that facilitates the inspection of the breast for irregularities that may represent cancerous tissue.

In the present invention, these and other objects are achieved by a process that comprises the step of first spraying the skin with a composition comprising water, witch hazel, less than 0.1% essential oil, the mixture being stabilized by an emulsifying agent. While the skin is still wet from the above composition, a second composition comprising a mixture of essential oils in a vegetable oil carrier base is gently massaged into the skin until absorbed.

This combined two-step process results in an improvement in skin tone and softness on all types of skin tissue, and aids in the physical examination of breast tissue. As the process is also effective in toning and firming breast tissue it promotes the regular daily use on breast tissue such that an overall message is effective in detecting potentially cancerous abnormalities at an early stage.

The invention will become more apparent from the following description of various embodiments.

### BRIEF DESCRIPTION OF DRAWINGS

FIG. 1 is a perspective view illustrating a treatment kit as one embodiment of the invention.

### DETAILED DESCRIPTION

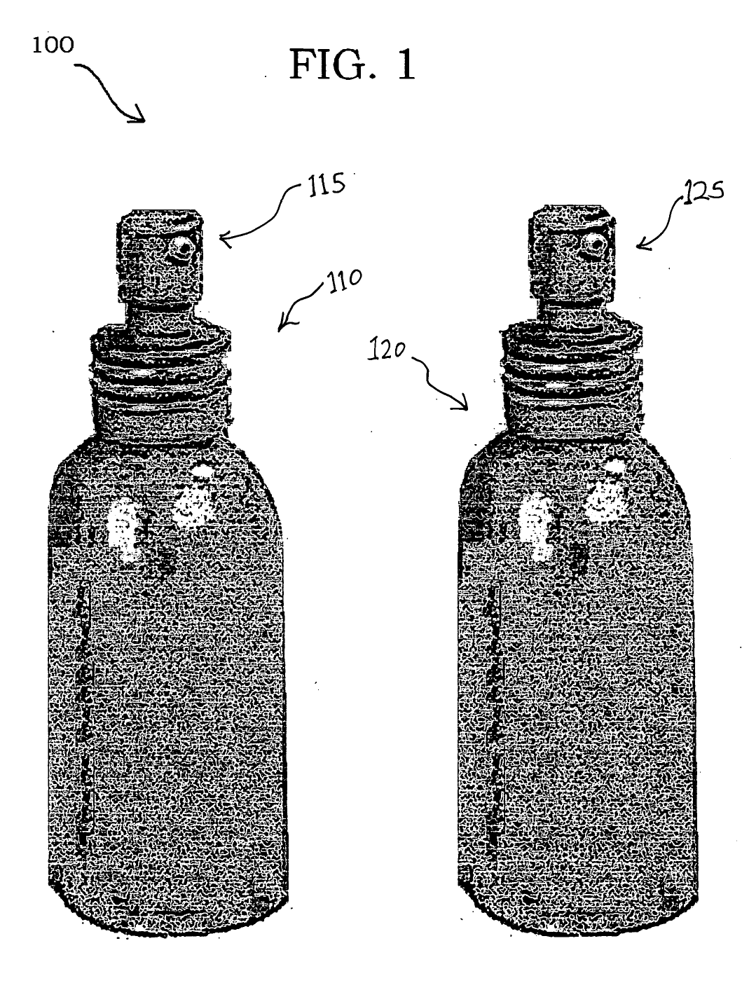

In accordance with one aspect of the present invention there is provided a kit, 100 shown in FIG. 1, containing ingredients for a 1^{st} and 2^{nd} stage treatment of the skin, and in particular breast tissue. The chemical composition for first stage of the treatment contains water, witch-hazel and up to about 3 vol. % essential oils and an emulsifying agent. A preferred chemical composition for the first stage of treatment is provided in TABLE I. In this preferred compositions the essential oil components comprise rose otto essential oil and lavender essential oil. The grapefruit seed extract is present in small quantities for its anti-septic properties. In preparing the Phase I composition, the coconut emulsifier and essential oil must be mixed together first before putting into the distilled water. To the extent that the compositions is varied, that is increasing or decreasing the total concentration of essential oil, the emulsifier concentration should be likewise adjusted such that 2 parts of emulsifier are mixed with 1 part of essential oil or the mixture of essential oils. Thereafter, the oil/emulsifier mixture is added to the water, or the mixture of water and witch hazel, and the composition is shaken, stirred or otherwise well mixed to homogenize the ingredients.

TABLE I
Phase I Composition

| PHASE I COMPONENT | Vol. % |
|---|---|
| DISTILLED WATER | 87.38% |
| WITCH HAZEL | 12.48% |
| ROSE OTTO | 0.029% |
| ESSENTIAL OIL | |
| LAVENDER | 0.014% |
| ESSENTIAL OIL | |
| COCONUT | 0.086% |
| EMULSIFIER | |
| GRAPEFRUIT SEED | 0.003% |
| EXTRACT | |

Witch Hazel is an astringent produced from the leaves and bark of the North American shrub Witch-hazel. Witch Hazel has been used externally on sores, bruises and swelling. The principal components of the extract are tannin, gallic acid, catechins, proanthocyanins, flavonoids (kaempferol, quercitin), essential oil (carvacol, eugenol, hexaenol), choline, saponins, and bitters. Distilled witch hazel, which contains no tannin, but may contain circa 14% alcohol may be used, as well as alcohol free distillates of witch hazels known as “Hydrosols”. It should be appreciated that as witch hazel is a natural product its odor may vary from batch to batch. Accordingly, depending on the olfactory quality of the witch hazel its percentage in the formulation may be varied accordingly, along with that of the essential oils to provide a consistently pleasing odor. Generally, the witch hazel should not exceed about ⅓ of the total volume, due to its astringent properties.

The chemical composition for second stage of the treatment contains one or more vegetable oils and up to about 3 volume (vol.) % essential oils. A preferred chemical composition for the second stage of treatment is provided in TABLE II. An alternative chemical composition for the second stage of treatment is provided in TABLE III.

TABLE II
Phase II Composition

| | | AS VOL. % OF | AS VOL. % OF |
|---|---|---|---|
| PHASE II COMPONENT | VOL. % | CARRIER OIL | ESSENTIAL OIL |
| GRAPESEED OIL | 77.016% | 77% | |
| OLIVE OIL | 10.809% | 11% | |
| SWEET ALMOND OIL | 10.809% | 11% | |
| JOJOBA OIL | 1.351% | 1% | |
| GRAPEFRUIT SEED EXTRACT | 0.001% | Less than 1% | |
| GERANIUM ESSENTIAL OIL | 0.004% | | 31% |
| CYPRESS ESSENTIAL OIL | 0.004% | | 31% |
| LEMON GRASS ESSENTIAL | 0.002% | | 13% |
| OIL | | | |
| CARROT SEED ESSENTIAL | 0.002% | | 13% |
| OIL | | | |
| CLARY SAGE ESSENTIAL OIL | 0.002% | | 12% |
| % OF CARRIER OIL | 99.986% | | |
| % OF ESSENTIAL OIL | 0.0141% | | |

TABLE III
Alternative Phase II Composition

| | | | AS VOL. |
|---|---|---|---|
| | | | % OF |
| | | AS VOL. % OF | ESSENTIAL |
| PHASE II COMPONENT | VOL. % | CARRIER OIL | OIL |
| GRAPESEED OIL | 77.015% | 77% | |
| OLIVE OIL | 10.809% | 11% | |
| SWEET ALMOND OIL | 10.809% | 11% | |
| JOJOBA OIL | 1.351% | 1% | |
| GRAPEFRUIT SEED | 0.001% | Less than 1% | |
| EXTRACT | | | |
| GERANIUM ESSENTIAL | 0.004% | | 27% |
| OIL | | | |
| CYPRESS ESSENTIAL | 0.004% | | 27% |
| OIL | | | |
| LEMON GRASS | 0.003% | | 22% |
| ESSENTIAL OIL | | | |
| CARROT SEED | 0.002% | | 12% |
| ESSENTIAL OIL | | | |
| CLARY SAGE | 0.002% | | 12% |
| ESSENTIAL OIL | | | |
| % OF CARRIER OIL | 99.984% | | |
| % OF ESSENTIAL OIL | 0.0158% | | |

The essential oils in the preferred embodiment of the Phase II composition include cypress, geranium rose, lemongrass, carrot seed, clary-sage, rose otto and true angustifolia. However, the Phase II compositions in TABLE II and III contain less than a total of 0.02 vol. % of essential oils, with the balance of the composition being the carrier oil. While the proportions of components of the carrier oil can vary, in the preferred embodiments of TABLE II and III it comprises about 77% by volume grapeseed oil, about 11% by volume oil, about 11% by volume sweet almond oil and about 1% by volume jojoba oil.

Another aspect of the invention is using the compositions listed in TABLE I and II or III. In the first step in the process the Phase 1 composition, is sprayed on the skin to be treated. Accordingly, another aspect of the invention is providing a treatment kit 100 in which the Phase 1 composition is contained in a bottle 110 having an attached spraying dispenser head 115. The container 110 holding the Phase I composition should be shaken or otherwise agitated prior to use.

The process of treatment and using the treatment kit 100 comprises a second step of applying the Phase II composition on the same area of skin tissue subject to the first treatment step and gently rubbing or messaging the thus combined compositions into the skin. The PHASE II composition is intended to be applied immediately after using the PHASE I composition, while skin is still damp from its application. Preferably, the Phase II composition is also sprayed on the skin. Accordingly, another aspect of the invention is providing within the treatment kit 100 the Phase II composition in a bottle 120 with a spraying dispenser head 125 attached. Whatever container holds the Phase II composition it is preferably shaken or otherwise agitated prior to use.

It has been discovered that the PHASE I AND PHASE II compositions work with each other to optimize the moisturizing and toning effects on the skin tissue. Not wishing to not be bound by theory, it is currently believed that the benefits of the 2-step process accrue because the combined treatment aids in the retention of the toning components of the Phase I compositions. It is also believed that the treatment with the Phase I composition makes the skin tissue more receptive to incorporating both the moisturizing oil components and the essential oil of the Phase II composition.

As a further aspect of the invention it has been discovered that the combination of the Phase I treatment, which provides a moist base, in combination with the carrier oil(s) in the Phase II composition lubricates the fingers to further aid in breast examination during the third stage in the application process. Again, not wishing to be bound by theory, it is also believed that the beneficial viscosity and lubricity of the skin in the Phase II process is caused in part by the aqueous nature of the Phase I treatment. Specifically, as the Phase I composition contain an emulsifying agent (coconut emulsifier) the phase II composition being an oil base will gradually emulsify with the phase I composition during the messaging action. This also extends the time before evaporation of absorption of the various components to allow examination of the entire breast under approximately the same conditions of lubricity. It is further believed that the immediate softening effects on the skin tissue aids in the detection of breast abnormalities.

Although the essential oil combination in the preferred embodiment of the Phase I compositions includes the essential oil of rose otto and true angustifolia, other essential oils that may be used or combined. These include oil of bitter almond, benzoin, rosewood, camomile, carrot, cedar, lemon, cypress, eucalyptus, clove, hyssop, lavender, lime, mace, nutmeg, bitter orange, oregano, palmarosa, patchouli, rosemary, sandalwood, sage. Whatever the combination of essential oils it is preferable that they be present in a concentration range from about 0.01 to 3 vol. %.

Thus, it should be appreciated that the carrier oils in the Phase II compositions are not merely inert ingredients, but also contribute to the immediate skin softening and provide the level of lubricity that aids in breast examination. In particular, the grape seed oil renders the Phase II combination non-greasy because it is readily absorbed into the skin. Further, as the grape seed oil has no odor, it dilutes odor of the more pungent carrier oils. Providing a non-greasy composition is important so that the user can dress immediately after the final application and messaging of their skin. It should be noted that sweet almond has no odor, but is known to add moisture to and lubricate the skin.

The olive oil is highly desired for its benefits to the skin conditioning process, while the grapefruit seed oil is desirable for its antiseptic properties. The quantity of olive oil is generally kept below about 15% because the strong odor may be objectionable to some consumers. However, to the extent that the selection of essential oils compliment or mask this strong odor, the concentration of olive oil can be increased upwards, so long as the viscosity and skin absorption properties are not radically changed from the preferred compositions.

As for alternative embodiments of the Phase II composition, it should be appreciated that the mixture and percentage of the carrier oil can be varied. For example, it may contain from about 70 to 95 vol. % grapeseed oil; from about 5 to 20% vol. olive oil (but preferably below 15 vol. %); from about 0 to 15% vol. sweet almond oil and from about 0.5 to about 5% vol. jojoba oil or a derivative thereof It may also include other vegetable oils that are readily absorbed by the skin.

Whatever the combination of essential oils, it is preferable that they be present in a concentration range from about 0.01 to 3 vol. %. As the carrot seed and clary sage essential oils are very strong, they should not together generally exceed about 0.005 vol. % of the total Phase II composition.

Alternatives to the essential oil combination in the preferred embodiment of the Phase II compositions include oil of bitter almond, benzoin, rosewood, camomile, cedar, lemon, cypress, eucalyptus, clove, hyssop, lavender, lime, mace, nutmeg, bitter orange, oregano, palmarosa, patchouli, rosemary, sandalwood, sage.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A kit for the treatment of the skin and breast tissue comprising:
a) a first container capable of applying a mist to the skin holding a fluid composition that comprises water, witch hazel, at least one essential oil and an emulsifying agent,
b) a second container holding a composition that comprises a mixture of vegetable oils and essential oils.

2. A kit for the treatment of the skin and breast tissue according to claim 1 wherein the at least one essential oil comprises up to about 3 volume % of the fluid in said first container.

3. A kit for the treatment of the skin and breast tissue according to claim 2 wherein at least 2 parts of emulsifying agent are mixed with a total of 1 part of essential oils before adding to the water or witch hazel that is to comprise the fluid composition held in said first container.

4. A kit for the treatment of the skin and breast tissue according to claim 1 wherein the vegetable oils in said second container comprises grapeseed oil and olive oil.

5. A kit for the treatment of the skin and breast tissue according to claim 5 wherein the vegetable oils in said second container further comprises jojoba oil.

6. A kit for the treatment of the skin and breast tissue according to claim 3 wherein the vegetable oils in said second container comprises grapeseed oil and olive oil.

7. A kit for the treatment of the skin and breast tissue according to claim 6 wherein the vegetable oils in said second container further comprises jojoba oil.

8. A composition of matter for treating skin and breast tissue, the composition comprising a first treatment solution that comprises:
a) water,
b) witch hazel,
c) up to about 3 volume % of an essential oil, and
d) an emulsifying agent.

9. A composition of matter for treating skin and breast tissue according to claim 8 wherein at least 2 parts emulsifying agent are mixed with a total of 1 part of essential oils before adding to the water or witch hazel.

10. A composition of matter for treating skin and breast tissue according to claim 8 comprising the essential oils of rose otto and lavender.

11. A composition of matter for treating skin and breast tissue according to claim 8, the composition comprising a second treatment solution that comprises:
a) from about 0.01 to about 3 volume % essential oils,
b) one or more vegetable oils as a carrier for the essential oils.

12. A composition of matter for treating skin and breast tissue according to claim 11 where the vegetable oil component comprises;
a) from about 5 to 20 volume % olive oil, and
b) from about 70 to 95 volume % grapeseed oil.

13. A composition of matter for treating skin and breast tissue according to claim 12 wherein the vegetable oil component further comprises from about 0 to 15 volume % sweet almond oil.

14. A composition of matter for treating skin and breast tissue according to claim 12 wherein the vegetable oil component further comprises from about 0.5 to 5 volume % jojoba oil.

15. A composition of matter for treating skin and breast tissue according to claim 13 wherein the vegetable oil component further comprises from about 0.5 to 5 volume % jojoba oil.

16. A process for treating skin, the process comprising the steps of:
a) providing a first composition that comprises water, witch hazel and at least one essential oil,
b) providing a second composition that comprises at least one vegetable oil and at least one essential oil,
c) spraying the first composition on the skin,
d) rubbing the second composition into the portion of the skin to which the first composition has been applied, before the first composition dries.

17. A process for treating skin according to claim 16 wherein the essential oils comprise up to about 3 volume % of the first composition.

18. A process for treating skin according to claim 16 wherein the second composition comprises grapeseed oil and less than about 20% olive oil.

19. A process for treating skin according to claim 18 wherein the composition of the second container further comprises jojoba oil.

20. A process for treating skin according to claim 16 wherein the first composition comprises the essential oils rose otto and lavender.
